(19) United States
(12) Patent Application Publication
(10) Pub. No.: US 2007/0078353 A1
(43) Pub. Date: Apr. 5, 2007

(54) Method and apparatus for removing baseline wander from an ECG signal

(75) Inventor: Alexander Holland, West Linn, OR (US)

Correspondence Address: WALL MARJAMA & BILINSKI, 250 SOUTH CLINTON STREET, SUITE 300, SYRACUSE, NY, 13202, US

(73) Assignee: Welch Allyn, Inc., Skaneateles Falls, NY (US)

(21) Appl. No.: 11/243,175

(22) Filed: Oct. 4, 2005

(51) Int. Cl. A61B 5/04

(52) U.S. Cl. 600/509

(57) ABSTRACT

According to one aspect of the invention, an improved ECG monitor includes a plurality of electrodes to be affixed to a patient's body to pick up ECG signals in an ECG signal band. The electrodes are electrically coupled to a plurality of input amplifiers. At least one analog to digital converter (“ADC”) is electrically coupled to the input amplifiers to digitize the ECG signals. A digital baseline wander filter has an internal finite impulse response (“FIR”) low pass filter characterized by a substantially trapezoidal impulse response. The baseline wander filter substantially removes a baseline wander signal component having a range of frequency components below the ECG signal band. The ECG waveform output signal is a baseline filtered ECG waveform representing the one or more of the ECG signals. The ECG waveform output signal from the improved ECG monitor is delayed less than 2 seconds from the ECG signals.

### FIELD OF THE INVENTION

This invention relates generally to a digital baseline wander filter for an ECG monitor and more particularly to a computationally efficient ECG baseline wander filter having minimal input to output signal delay.

### BACKGROUND OF THE INVENTION

An electrocardiogram (“ECG”) is a representation of the electrical signals generated by the heart muscle. Typical ECG apparatus derive one or more ECG waveforms by measuring small voltages that appear on pickup electrodes placed on the surface of a patient's body. The ECG monitoring apparatus typically presents the one or more ECG waveforms in the form of an electronic display, a printed page, and/or a strip chart print out. Some ECG monitors also provide various types of electronic signals for use by other equipment, such as a defibrillator, for synchronizing a therapeutic shock to a patient heart beat. It is also possible to integrate an ECG monitor with a defibrillator into a single fixed or portable instrument where the defibrillator is synchronized to the internally generated ECG monitor signal.

A common problem faced by all ECG monitors is to separate the actual heart muscle signals that represents the state of heart operation from unrelated factors that can distort the one or more ECG waveforms. Factors that can cause distortion in an ECG waveform include electrical noise in the environment, such as noise caused by nearby AC power wires in the walls and in other instruments, or electrical noise generated by electrical equipment, such as motors or fluorescent ceiling lamps. Other potential sources of electrical noise include radio noise, such as that caused by a two way radio or cellular phone. Still other factors can cause more slowly changing errors, such as a change in the conductivity of one or more electrodes on the surface of the skin. In addition to the above and many other patient distortion factors, patient breathing can also affect an ECG waveform.

Since the factors that can potentially affect the ECG waveform are relatively well understood, engineers can mitigate those effects by adding appropriate electronic filters to the ECG monitor signal paths. While electronic filters can be categorized by many different parameters, frequency is one of the most important defining features of a filter's performance. Most electrical signals can be represented as having primarily one frequency or as having a range or band of frequencies. For example, an ECG waveform mostly resides in a range of frequencies between about 0.5 Hz and 40 Hz, also known as the ECG signal band.

It is relatively easy to remove signals at very different frequencies from the range of frequencies of interest by using electronic filtering. For example, the radio frequency (“RF”) signals from police or fire department two-way radios is typically above 150 MHz, very remote from the frequency range of an ECG signal, and therefore such RF signals are easily filtered from the pickup electrode wires leading to a patient. Power line signals at 50 Hz or 60 Hz are very close to the high end of the ECG signal band and therefore are more difficult to remove. However, slightly more sophisticated techniques can be used that exploit the fact that such interfering signals are periodic in time at a well known or easily measured frequency, enabling suitable filtering to be performed.

Distorting or interfering factors to the ECG waveform that occur at relatively slow speeds are far more problematic. These interfering signals are generally far less predictable and can combine in ways such that a single interfering source cannot be isolated and measured. When viewed on a screen or paper printout, these slow interfering signals, if not properly filtered out of the ECG waveform, can cause the ECG signal to move vertically. This error is referred to as “baseline wander”. Factors that can cause baseline wander include changing skin resistance or movement of the ECG electrodes on the surface of the patient's skin and patient breathing or movement during ECG monitoring. Filtering of such effects is however possible, since the primary frequency components of these disturbances is typically in the range of 0.01 Hz to 0.5 Hz, or just below the ECG signal band. A problem in filtering baseline wander relates to the filter itself. The more effective a filter is, the more likely the filter itself introduces distortion in nearby frequency ranges. Therefore, a filter that is effective to a 0.5 Hz “cutoff frequency” at the edge of the ECG band, could itself cause distortion to the ECG waveform that potentially could result in erroneous interpretation by a clinician. For example, a clinician viewing the qualitative aspects of an ECG waveform including pulse widths, relative positions, and relative offset from a baseline needs to see an accurate representation of these parameters in order to diagnose correctly the condition of the patient's heart.

Digital filters are electronic filters that operate on a digital signal representing the electrical waveform of interest, such as an ECG waveform. Digital filters can be made in hardware with digital ICs or implemented in software typically running on a microcomputer embedded within an instrument, such as an ECG monitor. One advantage of digital filters is that they are less affected by natural environmental changes (temperature, humidity, etc.) and component drifts (resistor, capacitor, etc.) than earlier analog filters made directly from electronic hardware components. A digital representation of a signal results from sampling the signal, generally at fixed time intervals of a small fraction of a second. For example, an ECG signal can be made into a digital signal of digital numbers that represent the amplitude of the signal every two one thousandths of a second (a sample rate of 500 Hz). Digital filters are typically classified as having an infinite impulse response (“IIR”) or a finite impulse response (“FIR”), and can be further described by a corresponding “impulse response function”.

One such digital ECG baseline wander filter was described in U.S. Pat. No.6,280,391 to Olson, et al., hereinafter (“Olson”). Olson recognized that an IIR filter, while computationally efficient, was problematic for use as a baseline wander filter because an IIR filter would introduce significant phase distortion into the ECG waveform. Olson's solution was to employ two (concatenated) stages of FIR filters that together have a triangular impulse response. The problem is that Olson's baseline wander filter adds a long delay of several seconds from the actual occurrence of a particular heart beat to the corresponding output of ECG waveform data representative of that particular heart beat. It should be noted that a faster microcomputer would not improve the delay performance that is fundamentally related to the triangular impulse response and sample rate.

The long delay of Olson's baseline wander filter design is strictly a function of the number of samples of the ECG data that is required before a first point of filtered ECG waveform data can be generated. That is, to match the performance of this invention at any particular sampling rate, Olson's filter requires significantly more samples to generate each output point. Therefore, no matter how fast the computer running the filter algorithm is, Olson's filter must still wait for the required number of successive samples before it can generate the filtered ECG waveform output data. Since samples are only received at the ECG apparatus sample rate, more quickly processing the calculations related to each input sample can not improve the overall delay in the output ECG waveform.

As was previously discussed, it can be highly advantageous for an ECG monitor to be able to provide electronic signals to automatically synchronize a defibrillator to a patient's actual heart beat. Because a human heart beat is somewhat periodic and regular, in the most undemanding of applications it might be possible to very roughly synchronize medical instruments, even where ECG data is excessively delayed from the actual heart beat. For example, a slaved machine might predict the next occurrence of a heart beat by past measurements, thus achieving a sort of pseudo synchronization. The problem with this type of delayed synchronization is that the human heart beat is not perfectly periodic. Even in an ECG waveform corresponding to a normal healthy heart beat, there is some variation with breathing or momentary exertion. More problematic is that a slaved medical instrument, particularly a defibrillator, is most crucially needed in grossly abnormal situations. At such anomalous times, it is far more likely that variations in heart beat and shape of the ECG waveform might vary significantly from beat to beat resulting in incorrect synchronization or misfire of an administered therapeutic operation where the ECG waveform is greatly delayed from the actual heart operation it is measuring.

Delays are also problematic when a human must respond to an emergency. For example, where a clinician remotely monitors one or more patients, a response must be provided as quickly as possible to a patient whose ECG waveform shows them to be in heart failure. Every second gained can improve the odds of a favorable patient outcome.

While some apparatus can include both instruments in a single package minimizing the detrimental effect of ECG waveform delay, it is increasingly more convenient for instruments to communicate with one another over computer networks, especially including wireless networks. Unfortunately, such networks can introduce additional signal delays of one to two seconds or more. One problem is that existing digital baseline wander filters, such as Olson's filter, already introduce a delay of several seconds and are therefore less suitable for use where a network connection can add an additional second or two of delay between an ECG monitor and a defibrillator. What is needed is a digital baseline filter with a signal delay time below two seconds for more accurate synchronization with a defibrillator and for synchronizing medical instruments to an ECG monitor over a wired or wireless network.

Industry specifications for ECG monitors, such as ANSI/AAMI EC11, define maximum amounts of ECG signal distortion that an ECG monitor can introduce into an ECG waveform. Typically, an engineer designing a baseline wander filter for an ECG monitor works forward from a frequency response range and then checks the resulting design response by how the resulting signal varies with time (in the “time domain”) against the various time domain requirements of EC11 for compliance. Where a design does not comply with EC11, the design might be iterated in frequency response and then retested in the time domain until compliance is achieved. Therefore, what is also needed is a method to design a near optimal ECG digital baseline wander filter characteristic (as described by an impulse function and transfer function) directly from a time domain medical instrument specification such as EC11.

### SUMMARY OF THE INVENTION

According to one aspect of the invention, an improved ECG monitor includes a plurality of electrodes to be affixed to a patient's body to pick up ECG signals in an ECG signal band. The electrodes are electrically coupled to a plurality of input amplifiers. At least one analog to digital converter (“ADC”) is electrically coupled to the input amplifiers to digitize the ECG signals. A digital baseline wander filter is electrically coupled to the at least one ADC to receive the digitized ECG signals. The baseline wander filter has an internal finite impulse response (“FIR”) low pass filter characterized by a substantially trapezoidal impulse response. The baseline wander filter substantially removes a baseline wander signal component having a range of frequency components below the ECG signal band. The ECG waveform output signal is a baseline filtered ECG waveform representing the one or more of the ECG signals. The ECG waveform output signal from the improved ECG monitor is delayed less than 2 seconds from the ECG signals.

According to another aspect of the invention, a method to design an ECG baseline wander filter having near optimum minimal delay while meeting industry requirements for ECG monitors comprises the steps of providing a set of relevant parameters from an ECG monitor performance specification; converting the relevant parameters to impulse response constraints on a set of discrete signal equations for a finite impulse response filter; providing a transfer function for a filter architecture; and reducing the impulse response constraints to a final set of equations for the filter architecture, to determine the parameters defining a finite impulse response of the ECG baseline wander filter.

In accordance with yet another aspect of the invention, an improved digital baseline wander (restoration) filter includes a low pass filter to high pass filter digital architecture having a first signal path and a second signal path. The first signal path includes a gain and delay element (all pass filter) and the second signal path includes a cascade of two or more FIR low pass filters. The improvement is to the impulse response of the low pass filter in the form of a finite impulse response (“FIR”) that is substantially trapezoidal in shape. A digital input signal is coupled to the first and second signal paths. The digital input signal has a signal band of interest of frequencies above a frequency f_{c} and a baseline wander including frequencies below f_{c}. The baseline wander filter substantially removes a baseline wander signal component having frequency components below f_{c} and passes the signal band of frequencies above f_{c} to generate a baseline wander filtered output signal having only a signal band of frequencies substantially above f_{c}.

### BRIEF DESCRIPTION OF THE DRAWINGS

For a further understanding of these and objects of the invention, reference will be made to the following detailed description of the invention which is to be read in connection with the accompanying drawings, where:

FIG. 1 is a block diagram of an exemplary ECG monitor;

FIG. 2 shows a baseline wander digital filter architecture;

FIG. 3 shows the FIR filters of the invention implemented as IIR filters;

FIG. 4 shows the trapezoidal impulse response according to the invention;

FIG. 5 is a block diagram showing the steps to design a baseline wander filter directly from an industry ECG performance specification;

FIG. 6a shows a basic high pass from low pass filter architecture;

FIG. 6b shows an improved high pass from low pass filter architecture;

FIG. 7 shows an inferior triangular impulse response;

FIG. 8 shows an embodiment of the baseline wander digital filter architecture having more than two cascaded filters in the low pass filter path; and

FIG. 9 shows the impulse response of the exemplary baseline wander filter of FIG. 8.

### DETAILED DESCRIPTION OF THE INVENTION

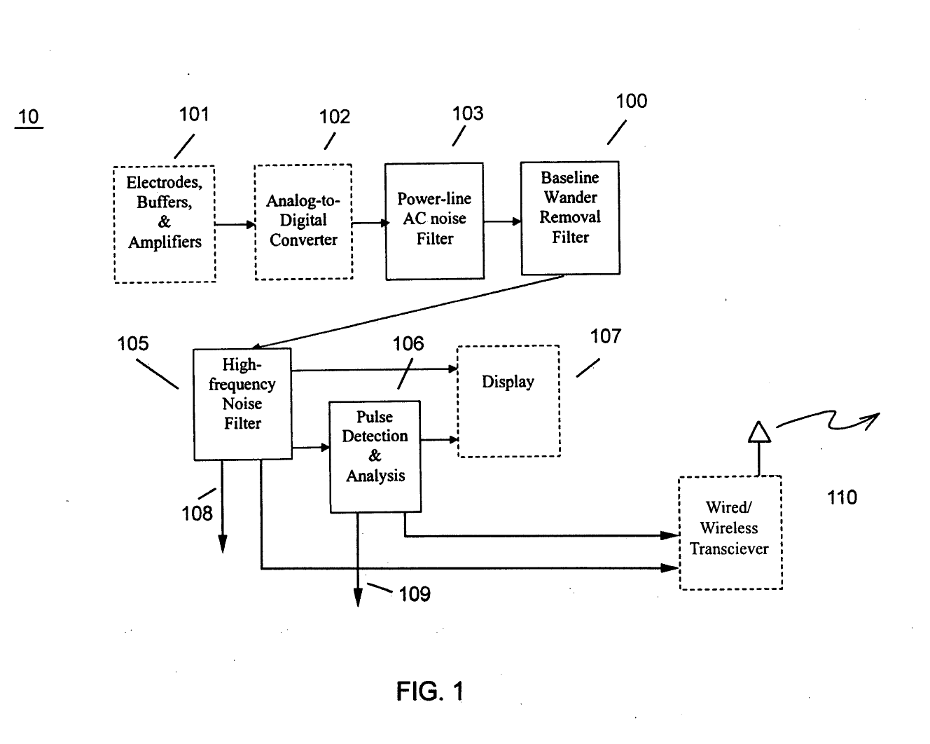

A block diagram of a typical ECG monitor 10 is shown in FIG. 1. A plurality of electrodes placed in physical and electrical contact with a patient's skin (not shown) are electrically coupled to analog buffers and amplifiers as shown by block 101. Analog to Digital Converter (“ADC”) 102 digitizes the analog electrode signals and converts them to digitally sampled discrete time signals. Power line AC noise filter 103 can reduce levels of AC power noise picked up by the electrodes, electrode connecting wires, and other sources of power line pickup. Baseline wander filter 100 reduces slowly changing errors on the ECG electrode signals that have frequency content generally below the ECG signal band. Some causes of baseline wander include changes in skin impedance due to physiological regulations such as sweating to maintain temperature, respiration, and changes in electrode-skin impedance due to a sudden or gradual loosening of the electrode, and front-end circuitry (blocks 101, 102) automated signal centering adjustments. Block 105 filters noise from above the ECG signal band, and block 106 performs ECG pulse detection and analysis routines, including detecting peaks to calculate heart rates and to make other qualitative measurements based on peak detection. Block 107 can display the filtered and processed ECG waveforms. Electrical signals in the form or digital waveform 108 and ECG waveform synchronization signals 109 representative of the filtered ECG waveforms can be further generated for use within a multifunction instrument, such a combined ECG monitor and defibrillator (not shown) or as a remote signal transmitted by wires and cables, over a wired network, or over a wireless connection 110. Block 110 can also be a wired or wireless transceiver. Filtered and/or otherwise processed ECG signals can be referred to in general as ECG waveform output signals. An ECG waveform output signal is defined herein as a baseline filtered ECG waveform or an ECG synchronization signal representing one or more of the ECG signals.

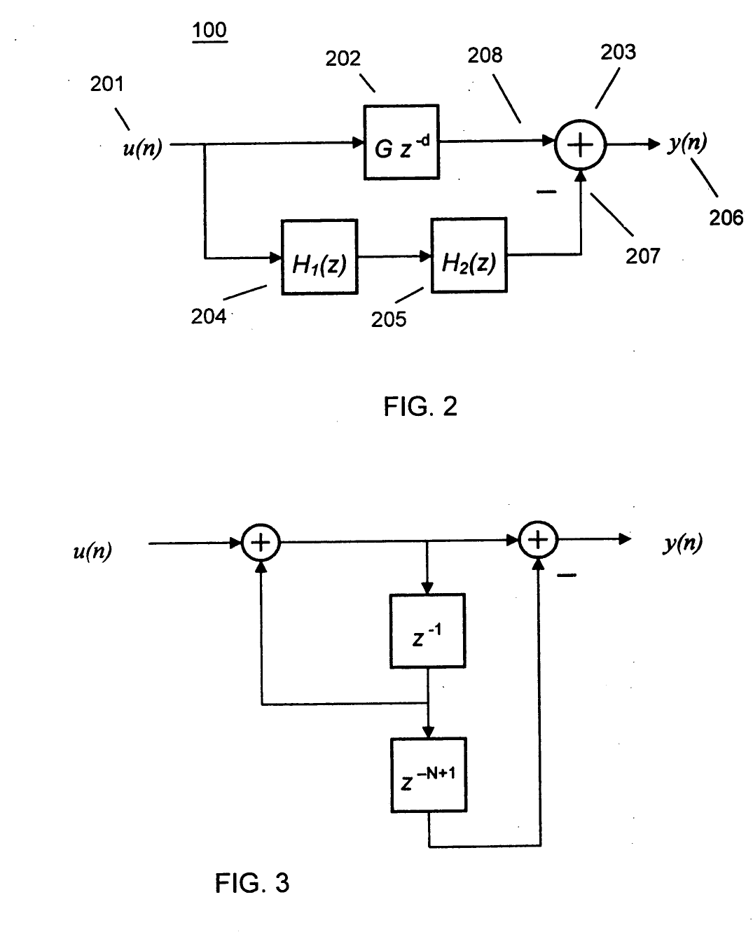

The inventive baseline wander filter architecture comprises two cascaded FIR filters as shown FIG. 2. Input signal u(n) 201 represents a discrete input ECG waveform to be baseline filtered. Signal 201 is split between two paths. The path through low pass filter transfer function H₁(z) 204 and low pass filter transfer function H₂(z) 205 produces a low pass filtered version of input signal 201 at input 207 to subtractor 203. The phase of the signal at 207 is delayed (d) from signal 201. The signal path through All Pass filter 202 is modified by gain (G) and delay (d) but the signal through this path is otherwise unmodified as a function of frequency. The primary function of block 202 is to delay the unfiltered signal 208 to match the net delay though filter sections 204 and 205 such that signal 207 can be subtracted from the corresponding points of input signal 201, thus phase matching signals 207 and 208. Those skilled in the art will recognize that output signal y(n) 206 is a high pass filtered version of input signal 201. For those less skilled in the art of digital filters, Appendix I introduces some of the basic relationships helpful to understand this high pass filter topology.

The inventive baseline wander filter employs two or more cascaded boxcar filters in the low pass filter path. A boxcar filter has a rectangular shaped impulse response; that is, it has a (finite) impulse response function where each output sample has the same value for a finite time period and has the value zero thereafter. Further, according to the inventive baseline wander filter, each boxcar filter can be implemented using the infinite impulse response (“IIR”) structure shown in FIG. 3. A baseline wander filter according to the invention can implement the two or more filters with the computational efficiency of the IIR structure with only two calculations are required per sample per filter, while still providing the linear phase response of an FIR filter. The IIR structure of FIG. 3 needs only 2 add/subtracts per sample per filter.

Computational efficiency alone, however does not solve the problem of long delays (d) presented by prior art transfer functions used in the architecture of filters 204 and 205 of FIG. 2. Once a computation has proceeded fast enough so as not cause an additional delay beyond the sample rate, no additional amount of computing speed can cause the output signal 206 to appear with less delay. This is a fundamental limitation of digital filters, that once the impulse function is fully defined, for a given sample rate, a fixed number of samples need to propagate through the digital filtering process. The filter requires this number of input samples to begin to generate meaningful filtered output data thus causing the delay d.

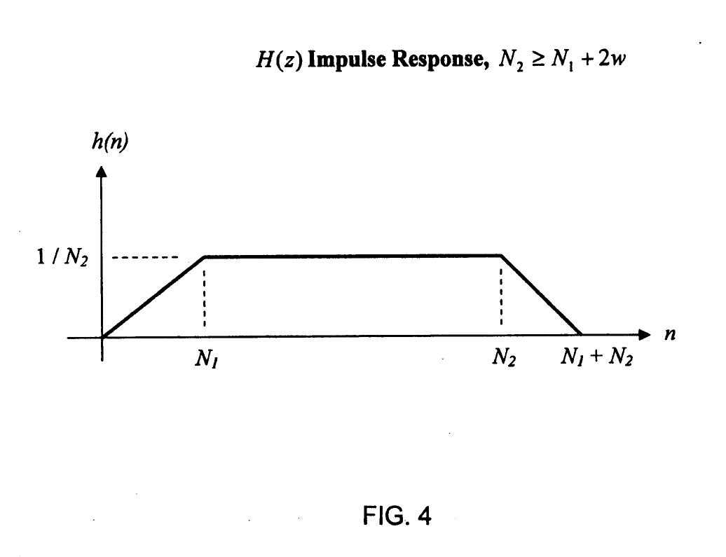

Another important improvement is that the applicant realized the prior art impulse functions, while providing adequate baseline filter functionality were far from optimal with respect to delay. It was realized that a substantially trapezoidal shaped impulse response as shown in FIG. 4, as opposed to the prior art triangular shaped transfer function provides a delay that is surprisingly almost one half of the delay caused the prior art baseline wander filter solution.

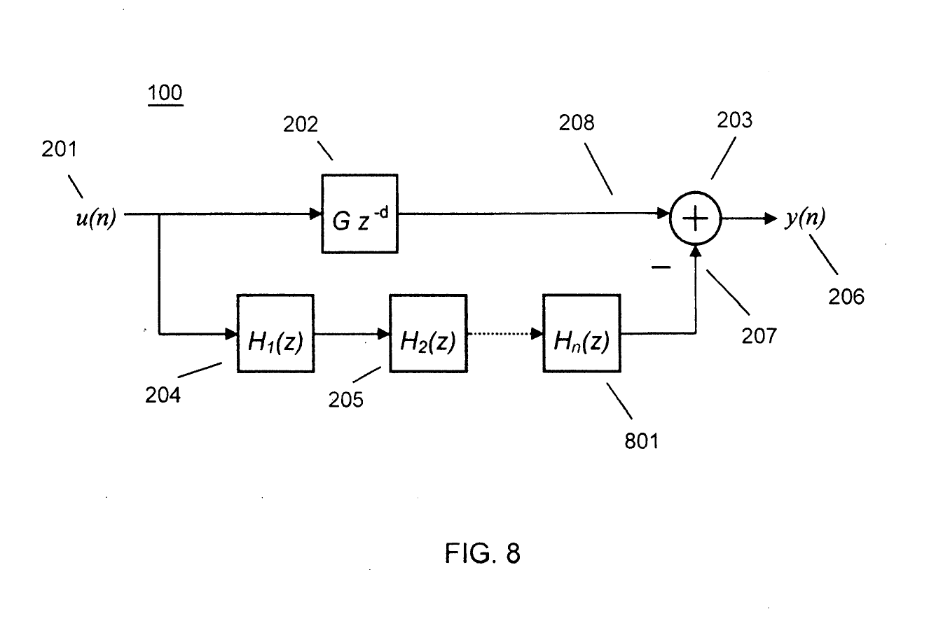

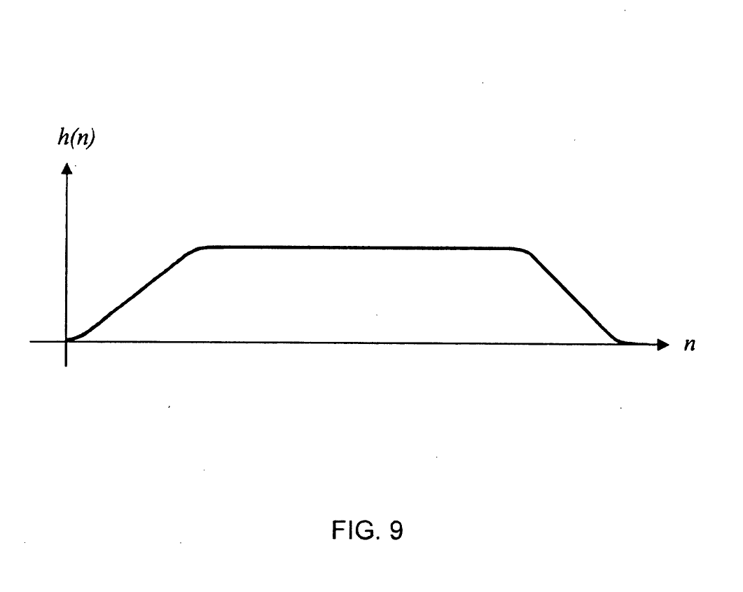

A “trapezoidal impulse response” is defined herein as an impulse response having a substantially trapezoidal shape, such as the trapezoidal impulse response of FIG. 9 with rounding of the corners. Typically the impulse response for two cascaded boxcar filters in the low pass filter path can be more nearly trapezoidal with relatively sharp comer transitions as shown in FIG. 4. In still other embodiments, adding a third or more additional boxcar filters 801 as shown in FIG. 8, generally with shorter delays, can cause the aforementioned rounding of the comers of the trapezoidal impulse response.

One skilled in the art could design other filter topologies that could also have a substantially trapezoidal impulse response. Such alternative topologies could implement the same impulse response and meet the same required input/output performance. Filter topologies, other than the preferred two or more boxcar filters in the low pass filter path, however, would likely differ in computational aspects such as the amount of required memory, sequence of arithmetic steps, and numerical roundoff.

The filter parameters of the near optimal transfer function were arrived at using a new approach to baseline wander filter design. Prior art base line filters were typically designed using frequency domain analysis (using Z transforms) and then cross checked for discrete signal (time domain) response. The time domain response would typically be checked for performance against an industry specification, such as the American National Standards Institute/Association for the Advancement of Medical Instrumentation (“ANSI/AAMI”) EC11 specification that defines the maximum amounts of ECG signal distortion that a filtering can introduce into an ECG waveform by an ECG monitor. The filter parameters could then be further iterated until the ECG performance specification was met. Applicant realized that a more efficient and more optimal method to design an ECG monitor base line wander filter can be achieved by converting the EC11 performance specifications directly into discrete signal design constraints from which filter coefficients and parameters could be directly obtained.

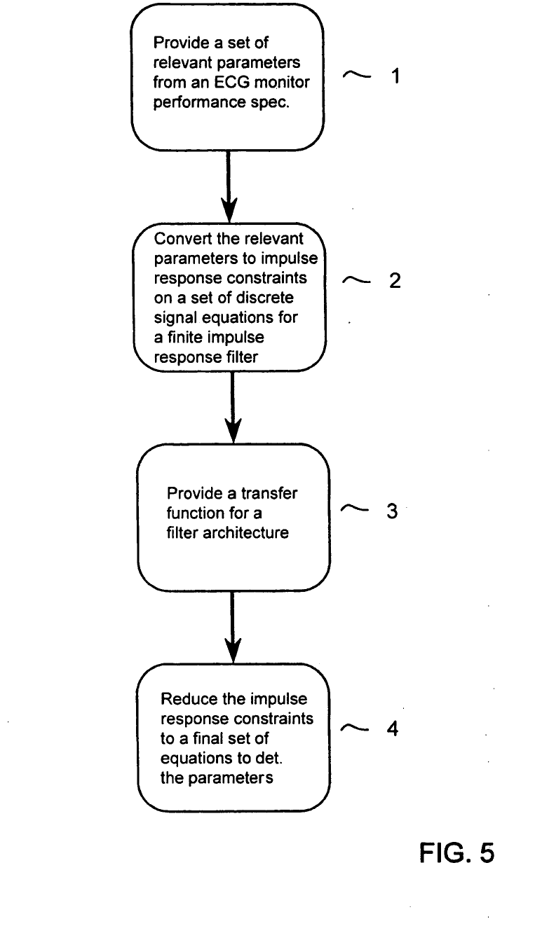

The inventive method as shown in FIG. 5 begins by defining and providing the relevant EC11 parameters in Step 1. The relevant parameters were found to be A, w, D, and S. As defined herein, A is the amplitude of an exciting finite test pulse, u(n), and w is the number of samples specifying the width of the test pulse. i.e.

$$u ( n ) = \{ \begin{matrix}A & n = 0 , Λ , w - 1 \\ 0 & n \geq w\end{matrix} \qquad Equation 1$$

D is the maximal allowed displacement error in the output waveform, y(n), due to u(n), while S is the maximal slope allowed in y(n). In Step 2, the relevant EC11 parameters are applied as constraints on a set of discrete signal equations for a response to the test pulse, as follows:

$$\begin{matrix}y ( n ) = {\sum }_{m = - ∞}^{∞} h ( n - m ) u ( m ) = A {\sum }_{m = 0}^{w - 1} h ( n - m ) < D \\ and & Equation 2 \\ \frac{\vert y ( n ) - y ( n - 1 ) \vert}{1 / {f}_{s}} = {Af}_{s} \vert {\sum }_{m = 0}^{w - 1} ( h ( n - m ) - h ( n - 1 - m ) ) \vert < S \\ or & Equation 3 \\ {\sum }_{m = 0}^{w - 1} h ( n - m ) < D / A \\ and & Equation 4 \\ \vert {\sum }_{m = 0}^{w - 1} ( h ( n - m ) - h ( n - 1 - m ) ) \vert < \frac{S}{{f}_{s} A} & Equation 5\end{matrix}$$

These design constraints on the filter impulse response, h(n), must hold for all sample times n outside of d−w/2<n<d+w/2; the response region outside the pulse. The constraints specify that the average of h(n) in any window of width w in a region outside the pulse must be less than D/A, and that the absolute value of the average of the difference between consecutive values in any window of width w in a region outside the pulse must be less than S/(fₛA). In Step 3, a transfer function for a proposed filter architecture is provided. In the embodiment of the exemplary baseline wander filter of the example, the equation 7 reflects one such architecture that yields the desired trapezoidal impulse response. In Step 4, assuming the filter architecture in FIG. 2 with H₁(z) and H₂(z) as expressed below in equation 7 of the example, and N₂≧N₁+2w, the above impulse response constraints reduce to:

$${N}_{2} \geq \frac{wA}{D} and {N}_{2} - 2 w \geq {N}_{1} \geq \frac{{f}_{s} wA}{{N}_{2} S} \qquad Equation 6$$

in the worst-case time window. The parameter N₂ independently determines the maximum displacement. As this parameter is increased, the bandwidth of the low-pass filter component is reduced, making the overall filter a less effective high-pass filter. Therefore N₂=wA/D can be set in order achieve the best possible bandwidth and meet the displacement constraint, and N₁ independently set to the lower bound of its constraint to minimize input to output delay. For any given sample rate fₛ, the resultant value of N₁ and N₂ fully define a near optimal baseline filter having a trapezoidal impulse response, as illustrated in FIG. 4. N₁ may be selected as slightly larger for the absolute minimum delay to minimize fixed-point arithmetic errors in an implementation on a particular microcomputer. Note that one could start with a different filter architecture and derive constraints on its parameters using Equations 4 and 5.

### EXAMPLE

An exemplary baseline wander filter according to the invention begins with the EC11 definitions, A=3 mV, w=fₛ/10, D=0.1 mV, and S=0.3 mV/s. The solutions of equations 4 and 5 are now applied the following transfer function:

$$H ( z ) = ( \frac{1}{{N}_{1}} \frac{1 - {z}^{- {N}_{1}}}{1 - {z}^{- 1}} ) ( \frac{1}{{N}_{2}} \frac{1 - {z}^{- {N}_{2}}}{1 - {z}^{- 1}} ) = \frac{( 1 + {z}^{- 1} + Λ + {z}^{- {N}_{1} + 1} ) ( 1 + {z}^{- 1} + Λ + {z}^{- {N}_{2 + 1}} )}{{N}_{1} {N}_{2}} \qquad Equation 7$$

The transfer function of Equation 7 represents a low-pass filter with a symmetrical, finite impulse response as required for linear phase. It is implemented as the concatenation of two FIR filters each implemented using an IIR topology in order to minimize computation. In addition, because each FIR filter has a boxcar impulse response of length N₁ and N₂respectively, the combined impulse response, h(n), is composed of straight lines that have either a slope of 0 or a fixed constant slope 1/N₁N₂ as shown in FIG. 4.
Assuming N₂≧N₁+2w and selecting a worst-case time window, the above impulse response constraints reduce to

$${N}_{2} \geq \frac{wA}{D} and {N}_{2} - 2 w \geq {N}_{1} \geq \frac{{f}_{s} wA}{{N}_{2} S} \qquad Equation 8$$

The parameter N₂ independently determines the maximum displacement. As this parameter is increased, the bandwidth of the low-pass filter component is reduced, making the overall filter a less effective high-pass filter, so N₂=wA/D is set to achieve the best possible bandwidth and meet the displacement constraint. For the EC11 parameters and fₛ=500, N₂≧1500 and 1400≧N₁≧500/3 ≈167. N₂=1500=wA/D and N₁=176 (N₁+N₂−1 must have odd length so that total delay d is an even number of samples). This meets the slope distortion spec with close to the minimum delay and transient/recovery time. N₁ can be further selected as a slightly larger number to minimize fixed-point arithmetic errors in our particular implementation. Note that this filter meets the distortion specifications while removing baseline wander of frequencies 0.3 Hz and below, less than 3.3 seconds initialization/recovery time (3 seconds with few percent error), and approximately 1.67 second input to output delay.

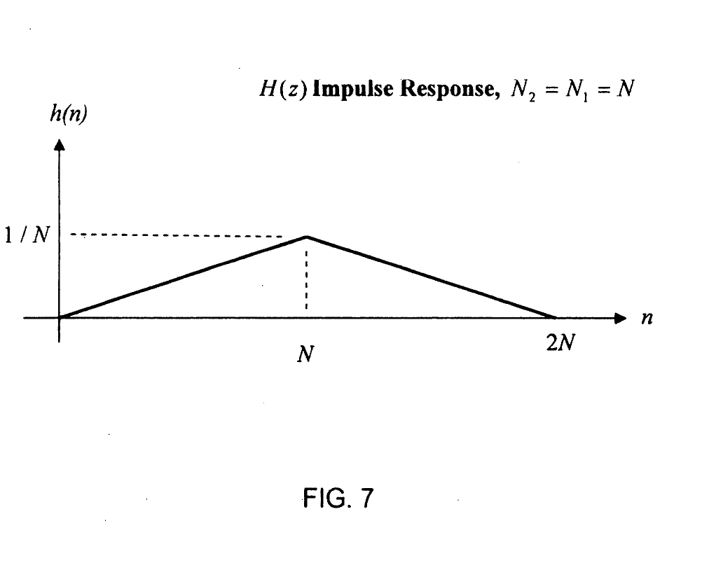

The inventive filter employing a trapezoidal impulse function exhibits almost an order of magnitude better performance than the filters of Appendix I and has almost one and one-half seconds faster input/output delay than a baseline wander filter using a triangular impulse function filter. For example, assume N₂<N₁+2w, e.g. N₂=N₁=N as shown by the triangular impulse function shown in FIG. 7. Selecting a worst-case time window (the maximum displacement constraint is now on a slope versus a flat region),

$$\begin{matrix}\frac{1}{{N}^{2}} {\sum }_{m = N - 2 w / 2}^{N - w / 2 - 1} < D / A & Equation 9 \\ \frac{2 {N}^{2}}{( 2 N - 2 w - 1 )} \geq \frac{wA}{D} \\ and & Equation 10 \\ {N}^{2} \geq \frac{{f}_{s} wA}{S} & Equation 11\end{matrix}$$

fₛ=500, N₂=N₁=N>1449 satisfies the EC11 constraints in this case. While a suitable solution strictly in terms of EC11, the time delay through the filter is now approximately 3 seconds, as compared to the far superior 1.67 second signal delay of an inventive baseline wander filter where N₁=167 and N₂=1501.

The shorter input to output signal delay can be important where the ECG waveform or a synchronization signal derived from the ECG waveform is used by another medical device, such as a defibrillator used to administer a therapeutic shock in the event of patient heart failure. Because the human heart beat period varies under normal circumstances due to breathing, exertion, excitement, and other physiological feedback mechanisms, and is particularly irregular in the case of trauma, a long input/out delay in measuring the ECG could compromise the performance of the defibrillator.

The shorter input to output signal delay can also be important where the ECG is monitored by a remote instrument or human observer. It is increasingly more convenient for instruments to communicate with one another over computer networks, especially including wireless networks. Because such networks can introduce additional signal delays of one to two seconds or more, long delays in signal filtering compound the problem of minimizing the overall delay to a remote observer, where precious seconds in being informed of an emergency may mean the difference between life and death. Long signal processing delays imply the need for more expensive network equipment to compensate.

The inventive digital baseline filter having a trapezoidal impulse response exhibits a significant improvement in input to output signal delay time in the range of one to two seconds. This improvement allows for more accurate synchronization to a defibrillator and for timely presentation of the ECG to a remote observer or instrument over a wired or wireless network. Therefore, the improved ECG monitor can be electrically connected to send ECG waveform signals over a cable, a wired network, local area network (“LAN”), a wireless network (such as IEEE 802.11 “WiFi” and other similar wireless local area networks (“WLAN”) systems), an optical link, an infrared link, an acoustic link, or an RF wireless link.

It should also be noted that the inventive baseline wander digital filter having a trapezoidal impulse response can be suitable for more general applications extending beyond ECG monitors. For example, the inventive filter is also suitable for use in any general digital baseline restoration operation in which signal frequencies lying just below a signal band of interest need to be removed (filtered) to restore a DC baseline.

While the present invention has been particularly shown and described with reference to the preferred mode as illustrated in the drawings, it will be understood by one skilled in the art that various changes in detail may be effected therein without departing from the spirit and scope of the invention as defined by the claims.

APPENDIX I-Filter Architecture to Make a High-pass Filter from a Low-pass Filter

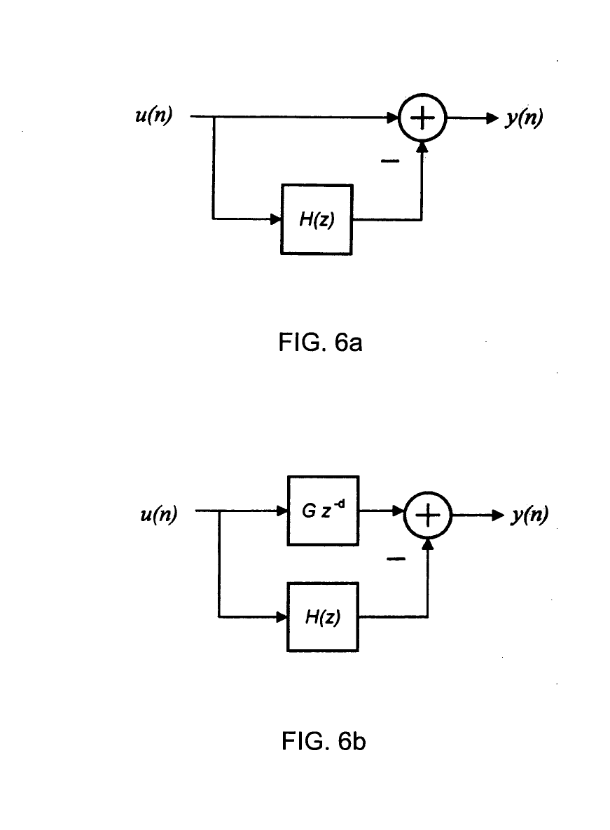

One technique to implement a high-pass filter (remove low frequencies, keep high frequencies) is to subtract a low-pass filtered signal (remove high frequencies, keep low frequencies) from the original signal as shown in FIG. 6a. The low-pass filter H(z) can have either an IIR or a FIR topology. In prior art approaches, this has been used with H(z) as a first-order IIR filter, which is computationally efficient, but has the problem of phase distortion limiting the lower cutoff frequency to an order of magnitude less than the FIR approach of the inventive baseline.

H(z) could also have the following transfer function:

$$H ( z ) = \frac{1}{N} \frac{1 - {z}^{- N}}{1 - {z}^{- 1}} \qquad Equation A1$$

Note that the transfer function of Equation A1 represents a low-pass filter that requires only 2 add/subtract operations and one division to calculate each output value. And if N is a power of 2, the division can be implemented by a logical shift right operation. Note also that this is an IIR architecture being used to implement an FIR filter,

$$H ( z ) = \frac{1}{N} \frac{1 - {z}^{- N}}{1 - {z}^{- 1}} = ( 1 + {z}^{- 1} + {z}^{- 2} + Λ + {z}^{- N + 2} + {z}^{- N + 1} ) / N \qquad Equation A2$$

The pole at 1 cancels in the numerator and denominator, and so this filter has both the computational efficiency of an IIR filter and the linear-phase property of an FIR filter. However, when used as in FIG. 6a, the overall high-pass filter loses the linear-phase property. Generally H(z) has gain, G, and time delay, d, between its input and output. FIG. 6b illustrates an improved architecture for creating a high-pass filter from a low-pass filter. If H(z) has linear phase, this architecture preserves the linear phase property.

Where H(z) is an IIR filter, there will also be improvement because the “matched delay” reduces phase distortion. The input/output time delay d of H(z) is defined to be the “center of gravity” of its impulse response, h(n), i.e.

$$d = \frac{{\sum }_{- ∞}^{∞} n h ( n )}{{\sum }_{- ∞}^{∞} h ( n )} \qquad Equation A3$$

The filter gain is defined as

$$G = {‖ h ( n ) ‖}_{1} = {\sum }_{n = - ∞}^{∞} \vert h ( n ) \vert \qquad Equation A4$$

which is motivated by the inequality
∥y(n)∥_{∞}≦∥u(n)∥_{∞}∥h(n)∥₁ Equation A5
that follows directly from the standard convolution operator describing the input to output mapping of a linear filter, where ∥·∥_{∞} is the infinity norm (peak or maximum absolute value of a signal) and ∥·n∥₁ is the 1-norm defined above. For H(z) as in Equation A1, G=1 and d=(N−1)/2. Note that N must be an odd number for the delay through the filter to be an integral number of samples. Where N is an even number, namely a power of 2, this division can be implemented with a right shift.

We claim:

1. An improved ECG monitor comprising:
a plurality of electrodes to be affixed to a patient's body to pick up ECG signals in an ECG signal band, the electrodes electrically coupled to a plurality of input amplifiers;
at least one analog to digital converter (“ADC”), the ADC electrically coupled to the input amplifiers to digitize the ECG signals;
a digital baseline wander filter electrically coupled to the at least one ADC to receive the digitized ECG signals, the baseline wander filter having an internal finite impulse response (“FIR”) low pass filter characterized by a substantially trapezoidal impulse response, the baseline wander filter to substantially remove a baseline wander signal component having a range of frequency components below the ECG signal band; and
an ECG waveform output signal, the ECG waveform output signal being a baseline filtered ECG waveform representing the one or more of the ECG signals, wherein the ECG waveform output signal from the improved ECG monitor is delayed less than 2 seconds from the ECG signals.

2. The ECG monitor of claim 1 wherein the baseline wander filter is of a low pass to high pass digital filter architecture.

3. The ECG monitor of claim 2 wherein the low pass to high pass digital filter architecture comprises a first signal path and a second signal path, the first signal path comprising a gain and delay element (all pass filter) and the second signal path comprising a cascade of two or more FIR low pass filters.

4. The ECG monitor of claim 3 wherein the substantially trapezoidal impulse results from two cascade boxcar filters in the second signal path or a trapezoidal impulse response with rounded corners resulting from more than two cascade boxcar filters in the second signal path.

5. The ECG monitor of claim 4 wherein the two or more FIR low pass filters are implemented as two FIR low pass filters using an infinite impulse response (“IIR”) computationally efficient filter topology.

6. The ECG monitor of claim 5 wherein the baseline wander filter transfer function is represented by the equation:
$$H ( z ) = ( \frac{1}{{N}_{1}} \frac{1 - {z}^{- {N}_{1}}}{1 - {z}^{- 1}} ) ( \frac{1}{{N}_{2}} \frac{1 - {z}^{- {N}_{2}}}{1 - {z}^{- 1}} ) = \frac{( 1 + {z}^{- 1} + Λ + {z}^{- {N}_{1} + 1} ) ( 1 + {z}^{- 1} + Λ + {z}^{- {N}_{2} + 1} )}{{N}_{1} {N}_{2}}$$

7. The ECG monitor of claim 6 wherein
$${N}_{2} \geq \frac{wA}{D} and {N}_{2} - 2 w \geq {N}_{1} \geq \frac{{f}_{s} wA}{{N}_{2} S} .$$

8. The ECG monitor of claim 6 wherein the sample rate fₛ is 500 Hz and N₂≧1500 and 1400≧N₁≧500/3≈167.

9. The ECG monitor of claim 1 further comprising a power line AC noise filter to remove power line noise from the ECG waveform signal output.

10. The ECG monitor of claim 1 further comprising a high frequency noise filter to remove high frequency noise from the ECG waveform signal output.

11. The ECG monitor of claim 1 further comprising a pulse detection and analysis function block to generate one or more ECG waveform synchronization signals.

12. The ECG monitor of claim 11 further comprising an electrical connection to send the ECG waveform signals to another device.

13. The ECG monitor of claim 12 wherein the electrical connection to send the ECG waveform signals is selected from the group of electrical connections consisting of a cable, a wired network, a wireless network, an optical link, an infrared link, an acoustic link, and an RF wireless link.

14. The ECG monitor of claim 13 wherein the device is a defibrillator.

15. A method to design an ECG baseline wander filter having near optimum minimal delay while meeting industry requirements for ECG monitors comprising the steps of:
providing a set of relevant parameters from an ECG monitor performance specification;
converting the relevant parameters to impulse response constraints on a set of discrete signal equations for a finite impulse response filter;
providing a transfer function for a filter architecture; and
reducing the impulse response constraints to a final set of equations for the filter architecture, to determine the parameters defining a finite impulse response of the ECG baseline wander filter.

16. The method of claim 15 wherein providing relevant parameters from an ECG monitor performance specification comprises providing relevant parameters from the American National Standards Institute/Association for the Advancement of Medical Instrumentation (“ANSI/AAMI”) EC11 specification.

17. The method of claim 16 wherein providing a set of relevant parameters comprises providing A, the amplitude of an exciting test pulse; w, the number of samples specifying the width of this pulse; D, the maximal allowed displacement error from the actual ECG waveform; and S, the maximal slope allowed at the end of the waveform.

18. The method of claim 17 wherein converting the relevant parameters to impulse response constraints comprises converting the relevant parameters to impulse response constraints as follows:
$${\sum }_{m = 0}^{w - 1} h ( n - m ) < D / A and \vert {\sum }_{m = 0}^{w - 1} ( h ( n - m ) - h ( n - 1 - m ) ) \vert < \frac{S}{{f}_{s} A}$$

19. The method of claim 18 wherein reducing the impulse response constraints to a final set of equations for the filter architecture, to determine the parameters comprises reducing the impulse response constraints to a final set of equations to determine the parameters for a concatenated filter topology.

20. The method of claim 19 wherein reducing the impulse response constraints to a final set of equations comprises reducing the impulse response constraints (for a baseline wander filter having two boxcar filters in a low pass filter path) to a final set of equations:
$${N}_{2} \geq \frac{wA}{D} and {N}_{2} - 2 w \geq {N}_{1} \geq \frac{{f}_{s} wA}{{N}_{2} S} .$$

21. An improved digital baseline wander (restoration) filter comprising:
a low pass filter to high pass filter digital architecture having a first signal path and a second signal path, the first signal path comprising a gain and delay element (all pass filter) and the second signal path comprising a cascade of two or more FIR low pass filters wherein the improvement is to the impulse response of the low pass filter in the form of a finite impulse response (“FIR”) that is substantially trapezoidal in shape; and
a digital input signal coupled to the first and second signal paths, the digital input signal having a signal band of interest of frequencies above a frequency f_{c} and a baseline wander including frequencies below f_{c}, the baseline wander filter to substantially remove a baseline wander signal component having a frequency components below f_{c} and to pass the signal band of frequencies above f_{c} to generate a baseline wander filtered output signal having only a signal band of frequencies substantially above f_{c}.

22. The digital baseline wander filter of claim 21 wherein f_{c} is in the range of 0.1 Hz to 0.9 Hz.

23. The digital baseline wander filter of claim 21 wherein the two FIR low pass filters are implemented using an infinite impulse response (“IIR”) computationally efficient filter topology.

24. The digital baseline wander filter of claim 21 wherein the baseline wander filter transfer function is represented by the equation:
$$H ( z ) = ( \frac{1}{{N}_{1}} \frac{1 - {z}^{- {N}_{1}}}{1 - {z}^{- 1}} ) ( \frac{1}{{N}_{2}} \frac{1 - {z}^{- {N}_{2}}}{1 - {z}^{- 1}} ) = \frac{( 1 + {z}^{- 1} + Λ + {z}^{- {N}_{1} + 1} ) ( 1 + {z}^{- 1} + Λ + {z}^{- {N}_{2} + 1} )}{{N}_{1} {N}_{2}}$$
